(19) United States
(12) Patent Application Publication
(10) Pub. No.: US 2023/0147190 A1
(43) Pub. Date: May 11, 2023

(54) TREATMENT FOR EPILEPTIC SEIZURES

(71) Applicant: Robert Andrew Bazzell, St. Charles, MO (US)

(72) Inventor: Robert Andrew Bazzell, St. Charles, MO (US)

(21) Appl. No.: 18/055,146

(22) Filed: Nov. 14, 2022

Related U.S. Application Data
(60) Provisional application No. 63/278,231, filed on Nov. 11, 2021.

(51) Int. Cl. A61K 31/05; A61K 31/352; A61K 47/44

(52) U.S. Cl. CPC A61K 31/05; A61K 31/352; A61K 47/44

(57) ABSTRACT

A method to treat epilepsy through administering a pharmaceutical composition comprising cannabidiol (CBD) and Δ9-tetrahydrocannabinol (THC) to the subject, wherein the ratio of CBD to THC in the pharmaceutical composition is between about 5:1 and about 1:1

### CROSS-REFERENCE TO RELATED APPLICATION

This application claims the benefit of priority of U.S. provisional application No. 63/278,231, filed 11 Nov. 2021, the contents of which are herein incorporated by reference.

### BACKGROUND OF THE INVENTION

The present invention relates to treatments or epilepsy and, more particularly, a treatment for uncontrolled, intractable epileptic seizures and a method of preventing sudden unexpected death in epilepsy (SUDEP).

Epilepsy is a group of non-communicable neurological disorders characterized by recurrent epileptic seizures. Anti-epileptic drugs (AED) are currently available for controlling two-thirds epileptic seizures. In other words, current treatments are unable to control approximately one-third of epileptic seizures. Individuals who suffer from these intractable seizures or drug-resistant seizures, and the people that care for them, are constantly facing the existential threat of sudden unexpected death in epilepsy (SUDEP).

In the United States, the FDA has indicated only one brand of prescription cannabidiol called “Epidiolex” for the treatment of seizures in people one year of age and older. While Epidiolex™ treatment is generally well tolerated, it is associated with minor adverse effects, such as gastrointestinal upset, decreased appetite, lethargy, sleepiness, and poor sleep quality.

Accordingly, only very recent prior art even delves into treating epileptic seizures with specific ratios of CBD to THC—and they are all teaching ratios of 10:1 and higher: U.S. published patent application US20220087951A1 (Knappertz) teaches a method of treating seizures in a patient in need thereof, comprising administering to the patient cannabidiol (CBD) drug substance having a purity of at least 95% (w/w) CBD, wherein the patient is administered a starting dose of CBD of 5 mg/kg/day; U.S. published patent application US20210121435A1 (O'Hearn, et al.) teaches a method for treating seizures in a subject having epilepsy, the method comprising administering a pharmaceutical composition comprising cannabidiol (CBD) and Δ⁹-tetrahydrocannabinol (THC) to the subject, wherein the ratio of CBD to THC in the pharmaceutical composition is between about 14:1 and about 17:1; and U.S. published patent application US20200215022A1 (Jacobson, et al.) teaches a pharmaceutical composition comprising cannabidiol (CBD) and delta-9-tetrahydrocannabinol (THC) at a ratio of from about 40:1 to about 60:1.

The inventor has a child, who at the age of 14-months, experienced an abrupt onset of hundreds of absence seizures a day that developed into mixed epileptic seizure types, including absence, focal, atonic, myoclonic-atonic, tonic, and eventually a 10-hour status epilepticus seizure. The Inventor was told his child would die in her sleep by the age of 3 from SUDEP and no medication will be able to treat her. AED did not work, neither did CBD alone. Keppra™ which reduced the frequency of the seizures but could not fully control them, also had side effects of nausea and “Keppra rage”. Upon clinical treatment of medical cannabis extract of 1:1 CBD to THC ratio when given along with the Keppra, the remaining uncontrolled seizures were reduced to the most recent EEG showing just one short brief seizure compared to the hundreds a day she was having. The side effects of the Keppra were also gone and the child gained the ability to walk and talk again within a week. The child is seven years old now and has developed normally.

As can be seen, there is a need for a teaching in a direction away from the prior art to a method to treat epilepsy through administering a pharmaceutical composition comprising cannabidiol (CBD) and Δ9-tetrahydrocannabinol (THC) to the subject, wherein the ratio of CBD to THC in the pharmaceutical composition is between about 5:1 and about 1:1, thereby providing drug-resistant epileptics a better quality of life, as well producing less side effects than experienced from AED.

### SUMMARY OF THE INVENTION

In one aspect of the present invention, a method for treating seizures in a subject having epilepsy, the method comprising administering a pharmaceutical composition comprising cannabidiol (CBD) and Δ9-tetrahydrocannabinol (THC) to the subject, wherein the ratio of CBD to THC in the pharmaceutical composition is between about 5:1 and about 1:1.

In another aspect of the present invention, the method for treating seizures in a subject having epilepsy includes, wherein the ratio of CBD to THC in the pharmaceutical composition is 3:1, wherein the ratio of CBD to THC in the pharmaceutical composition comprises an extract from a whole plant material, wherein the whole plant material are or is taken from a grown plant, wherein the pharmaceutical composition is administered as a dose of about 0.025 milligrams (mg) per kilogram (kg) of body weight per day for two weeks; and then a dose of about 0.050 mg/kg/day for a third week, wherein the pharmaceutical composition is administered as a dose of about 0.075 mg/kg/day after one month; and further including mixing the pharmaceutical composition with a carrier and subjecting the mixture to a heat source.

In yet another aspect of the present invention, a pharmaceutical composition comprising cannabidiol (CBD) and delta-9-tetrahydrocannabinol (THC) at a ratio of from about 5:1 to about 1:1.

These and other features, aspects and advantages of the present invention will become better understood with reference to the following drawings, description, and claims.

### BRIEF DESCRIPTION OF THE DRAWINGS

FIG. 1 is a schematic view of an exemplary embodiment of the present invention.

FIG. 2 is a flow chart of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, an embodiment of the present invention provides a method to treat epilepsy through administering a pharmaceutical composition comprising cannabidiol (CBD) and Δ9-tetrahydrocannabinol (THC) to the subject, wherein the ratio of CBD to THC in the pharmaceutical composition is between about 5:1 and about 1:1

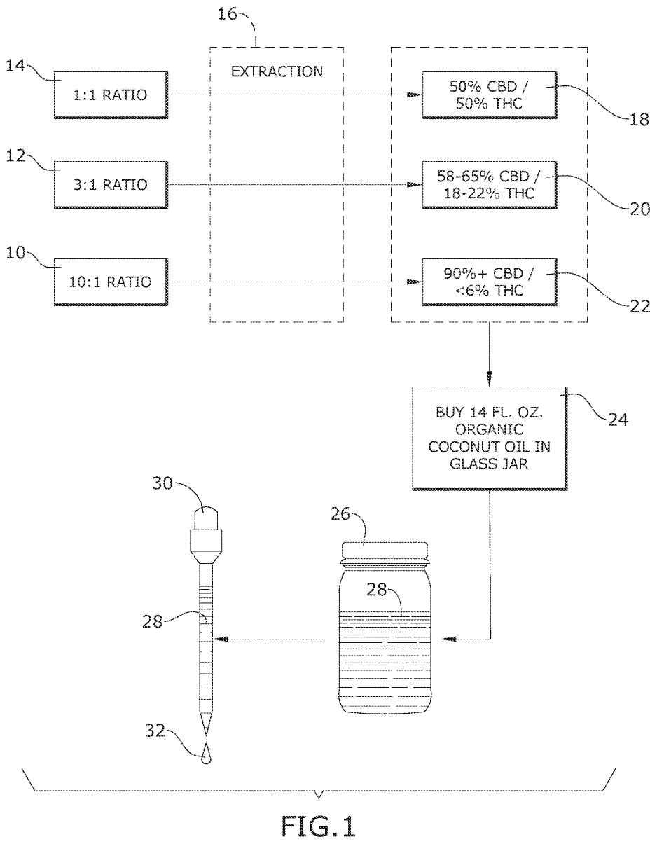

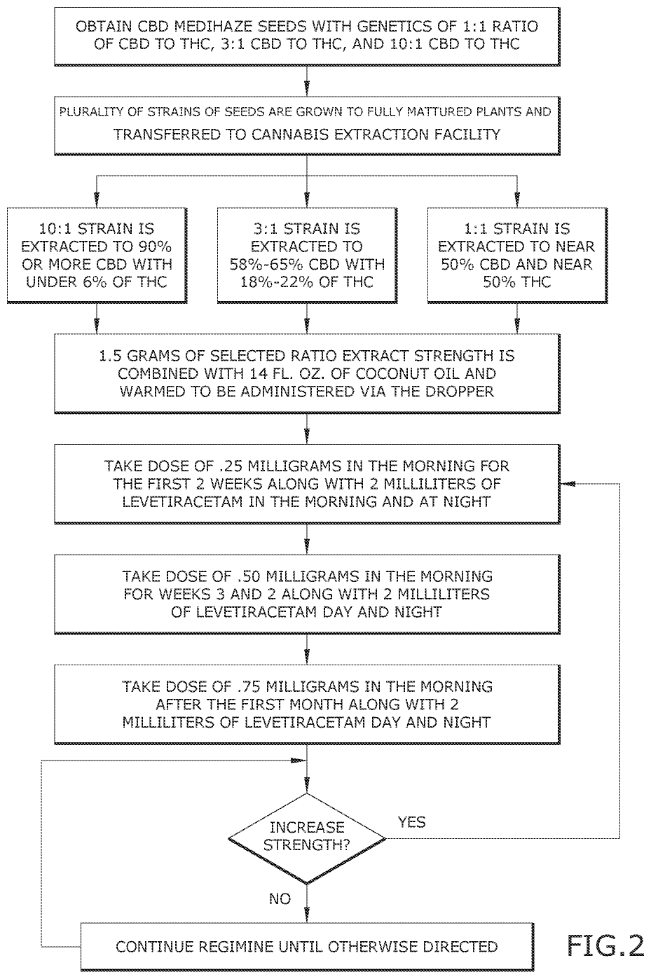

Referring now to FIGS. 1 and 2, the present invention may include a pharmaceutical composition comprising cannabidiol (CBD) and delta-9-tetrahydrocannabinol (THC) at a ratio of from about 5:1 to about 1:1, and a method of administering the treatment.

Cannabinoid medicines are preferrable sourced from whole plant material. Accordingly, the present invention embodies and contemplates a plurality of methods of providing pharmaceutical composition comprising CBD and THC at a ratio of from about 5:1 to about 1:1, through processing of the whole plant material. Thus, even though the below processing steps utilize extraction from plant parts of the cannabis flower/plant—including but necessarily grown plant material from a mature plant—may be used during extraction if the resulting pharmaceutical composition has a CBD:THC ratio of from about 5:1 to about 1:1.

One embodiment of the present invention includes the following steps:
(1) Procuring whole plant material with CBD with the genetics of 1:1 ratio of CBD to THC for (a first ratio 14); 3:1 CBD to THC (a second ratio 12); 5:1 CBD to THC; and/or 10:1 CBD to THC at (a third ratio 10). In certain embodiments, the whole plant is grown from CBD Medihaze™ seeds.
(2) Extracting 16 from whole plant material the above ratios: first the highest CBD plant may have a extracted CBD to THC ratio of 90%+CBD with under 6% THC (a third yield 18); the another extract may be the 5:1 ratio and it may have 14%-19% THC to 80-86% CBD. The third extract (a second yield 20) may be the 3:1 ratio and it may have 18%-24% THC to 58-65% CBD. The 1:1 extracted ratio (a first yield 22), may be extracted and as close to 50% THC to 50% CBD as possible.
(3) Introducing (via a container 26) approximately 1.5 grams of the given cannabis extract into approximately 14 fluid ounce full-circle organic refined coconut oil 24 or an equivalent carrier substance.
(4) Administering the resulting extract solution (subject to hydrolysis, crystallization, conversion to fluid form via a carrier subject to heat) 28 via a drop 32 through a dropper 30. In certain embodiments, an administrator may start with about 0.25 milligrams (mg) daily (in the mornings) for the first two weeks (for a child who weighs approximately 10 kilograms) and then about 0.50 mg for weeks three and four for this 10-kg child, and as high as about 0.75 mg to be safe after the first month of taking the cannabis extract. If you move up in THC level extracts, start back over at about 0.25 mg and work your way up again the same way you just did.
(5) Administering two milliliters (mL) of Levetiracetam, morning and at night.

The extraction process may contain a hydrolysis step (e.g., hydrolysis of (±)-1-m-nitrobenzenesulfoanate-6a,10a-trans-Δ9-tetrahydrocannabinol with NaOH in aqueous methanol to provide (±)-Δ9-THC). The extraction process may include a subsequent crystallization step of the resultant of the hydrolysis by way of using a non-polar organic solvents, including (but not limited to) using aliphatic (C4-C10)hydrocarbons such as butane, pentane, hexane, heptane, octane, nonane, decane, including straight-chained aliphatic hydrocarbons, branched aliphatic hydrocarbons and cyclic aliphatic hydrocarbons, or any mixture thereof. The extraction process may include other structurally changing steps.

The cannabis extract may be dropped in coconut oil and heated up to an oil form, thereby changing the structure of what was extracted from the whole plant. Coconut oil is the preferred transporter of cannabinoids more so than other oils tested on the market.

The daily dose for a child of 10 kg may be about 0.025 mg-0.075 mg/kg (body weight).

The Levetiracetam and cannabis are both anti-seizure medications. The Levetiracetam may be given twice a day, once in the morning and again at night.

The relatively high percentage of CBD mitigates negative side effect of THC away (e.g., anxiety or other adverse psychotomimetic effects) and works primarily as an anti-convulsant, though 90% of the time THC is an anti-convulsant. The coconut oil is the transporter of the cannabis extract as well as having medicinal properties of its own.

Also, the present invention embodies extracted medicinal ratio of CBD to THC with the right plant species to help intractable epileptics have relief.

As used in this application, the term “about” or “approximately” refers to a range of values within plus or minus 10% of the specified number. And the term “substantially” refers to up to 80% or more of an entirety. Recitation of ranges of values herein are not intended to be limiting, referring instead individually to all values falling within the range, unless otherwise indicated, and each separate value within such a range is incorporated into the specification as if it were individually recited herein.

It should be understood, of course, that the foregoing relates to exemplary embodiments of the invention and that modifications may be made without departing from the spirit and scope of the invention as set forth in the following claims.

### REFERENCES

Throughout this application, various references describe the state of the art to which this invention pertains. The disclosures of these references are hereby incorporated by reference into the present disclosure.
Bernard S. Chang, Md. Cannabidiol and Serum Antiepileptic Drug Levels: The ABCs of CBD With AEDs. Epilepsy Curr. 2018 January-February
Laurel P Gibson, Hollis C Karoly, Jarrod M Ellingson, Jost Klawitter, Cristina Sempio, Julia E Squeri, Angela D Bryan, L Cinnamon Bidwell, Kent E Hutchison. Effects of cannabidiol in cannabis flower: Implications for harm reduction. Addict Biol. 2022 January; 27
Ralph Karlerstuart, A. Turkanis Analysis, Metabolism, Cellular Responses, Reproduction and Brain. Pages 619-641. 1979
Use of Cannabis Flowers with Nearly Equal Ratios of THC and CBD Associated with Greater Overall Subjective Effects. NORML Jan. 13, 2022

What is claimed is:

1. A method for treating seizures in a subject having epilepsy, the method comprising administering a pharmaceutical composition comprising cannabidiol (CBD) and Δ9-tetrahydrocannabinol (THC) to the subject, wherein the ratio of CBD to THC in the pharmaceutical composition is between about 5:1 and about 1:1.

2. The method of claim 1, wherein the ratio of CBD to THC in the pharmaceutical composition is 1:1.

3. The method of claim 1, wherein the ratio of CBD to THC in the pharmaceutical composition comprises an extract from a whole plant material.

4. The method of claim 1, wherein the pharmaceutical composition is administered as a dose of about 0.025 milligrams (mg) per kilogram (kg) of body weight per day for two weeks; and then a dose of about 0.050 mg/kg/day for a third week.

5. The method of claim 4, wherein the pharmaceutical composition is administered as a dose of about 0.075 mg/kg/day after one month.

6. The method of claim 5, further including mixing the pharmaceutical composition with a carrier and subjecting the mixture to a heat source.

7. The method of claim 6, wherein the carrier is coconut oil.

8. A pharmaceutical composition comprising cannabidiol (CBD) and delta tetrahydrocannabinol (THC) at a ratio of from about 5:1 to about 1:1.

9. The composition of claim 8, wherein the CBD:THC ratio is from about 1:1.

10. The composition of claim 9, wherein the composition comprises an extract from a whole plant material.

11. The composition of claim 10, wherein the composition comprises a carrier subject to heat.

12. The composition of claim 11, wherein the carrier is coconut oil.
